(19) United States
(12) Plant Patent Application Publication
(10) Pub. No.: US 2017/0034976 P1
(43) Pub. Date: Feb. 2, 2017

(54) Calibrachoa plant named 'Duecalindapu'

(50) ;

(71) Applicant: Tobias Dummen, Rheinberg (DE)

(72) Inventor: Tobias Dummen, Rheinberg (DE)

(73) Assignee: DUMMEN GROUP B.V., De Lier (NL)

(21) Appl. No.: 14/756,084

(22) Filed: Jul. 29, 2015

(51) Int. Cl. A01H 5/00

(52) U.S. Cl. PLT/413

(57) ABSTRACT

A new and distinct cultivar of Calibrachoa plant named ‘Duecalindapu’, characterized by its compact, semi-upright and outwardly spreading to trailing and decumbent plant habit; freely branching habit; early and freely flowering habit; large velvety dark purple-colored flowers; and good garden performance.

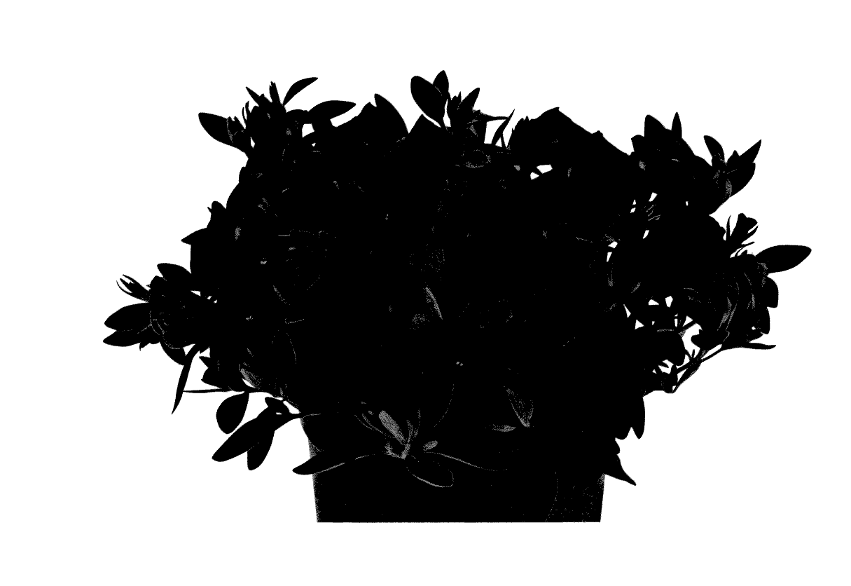

### BOTANICAL DESIGNATION

Calibrachoa sp.

### CULTIVAR DENOMINATION

‘DUECALINDAPU’

### BACKGROUND OF THE INVENTION

The present invention relates to a new and distinct cultivar of Calibrachoa plant, botanically known as Calibrachoa sp. and hereinafter referred to by the name ‘Duecalindapu’.

The new Calibrachoa plant is a product of a planned breeding program conducted by the Inventor in Rheinberg, Germany. The objective of the breeding program is to create new compact Calibrachoa plants with large and uniquely attractive flowers.

The new Calibrachoa plant originated from a cross-pollination made by the Inventor in June, 2012 in Rheinberg, Germany of a proprietary selection of Calibrachoa sp. identified as code number A10-3323-002, not patented, as the female, or seed, parent with a proprietary selection of Calibrachoa sp. identified as code number A11-1859-003, not patented, as the male, or pollen, parent. The new Calibrachoa plant was discovered and selected by the Inventor as a single flowering plant from within the progeny of the stated cross-pollination in a controlled greenhouse environment in Rheinberg, Germany in May, 2014.

Asexual reproduction of the new Calibrachoa plant by vegetative terminal cuttings in a controlled greenhouse environment in Rheinberg, Germany since June, 2014 has shown that the unique features of this new Calibrachoa plant are stable and reproduced true to type in successive generations.

### SUMMARY OF THE INVENTION

Plants of the new Calibrachoa have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Duecalindapu’. These characteristics in combination distinguish ‘Duecalindapu’ as a new and distinct Calibrachoa plant:
1. Compact, semi-upright and outwardly spreading to trailing and decumbent plant habit.
2. Freely branching habit.
3. Early and freely flowering habit.
4. Large velvety dark purple-colored flowers.
5. Good garden performance.

The new Calibrachoa can be compared to plants of the female parent selection. Plants of the new Calibrachoa differ primarily from plants of the female parent selection in flower color as plants of the female parent selection have darker purple-colored flowers. In addition, plants of the new Calibrachoa are more freely branching than plants of the female parent selection.

The new Calibrachoa can be compared to plants of the male parent selection. Plants of the new Calibrachoa differ primarily from plants of the male parent selection in plant size as plants of the new Calibrachoa are more compact than plants of the male parent selection. In addition, plants of the new Calibrachoa have smaller flowers than plants of the male parent selection.

Plants of the new Calibrachoa can also be compared to plants of Calibrachoa sp. ‘Duealmidpu’, disclosed in U.S. Plant Pat. No. 23,717. In side-by-side comparisons, plants of the new Calibrachoa differed primarily from plants of ‘Duealmidpu’ in the following characteristics:
1. Plants of the new Calibrachoa were more compact than plants of ‘Duealmidpu’.
2. Plants of the new Calibrachoa and ‘Duealmidpu’ differed in flower color as plants of ‘Duealmidpu’ had lighter purple-colored flowers.

### BRIEF DESCRIPTION OF THE PHOTOGRAPH

The accompanying colored photograph illustrates the overall appearance of the new Calibrachoa plant showing the colors as true as it is reasonably possible to obtain in colored reproductions of this type. Colors in the photograph may differ slightly from the color values cited in the detailed botanical description which accurately describe the colors of the new Calibrachoa plant. The photograph is a side perspective view of a typical flowering plant of ‘Duecalindapu’ grown in a container.

### DETAILED BOTANICAL DESCRIPTION

The aforementioned photograph and following observations, measurements and values describe plants grown during the summer in 10.5-cm containers in a glass-covered greenhouse in Rheinberg, Germany and under cultural practices typical of commercial Calibrachoa production. During the production of the plants, day and night temperatures averaged 18° C. and light levels averaged 4,500 lux. Rooted young plants were pinched one time three weeks after planting and were 13 weeks old when the photograph and description were taken. In the following description, color references are made to The Royal Horticultural Society Colour Chart, 2001 Edition, except where general terms of ordinary dictionary significance are used.
Botanical classification: Calibrachoa sp. ‘Duecalindapu’.
Parentage:
   Female, or seed, parent.—Proprietary selection of Calibrachoa sp. identified as code number A10-3323-002, not patented.
   Male, or pollen, parent.—Proprietary selection of Calibrachoa sp. identified as code number A11-1859-003, not patented.
Propagation:
   Type.—By vegetative cuttings.
   Time to initiate roots, summer.—About five days at temperatures about 20° C.
   Time to initiate roots, winter.—About seven days at temperatures about 20° C.
   Time to produce a rooted young plant, summer.—About three weeks at temperatures about 20° C.
   Time to produce a rooted young plant, winter.—About four weeks at temperatures about 20° C.
   Root description.—Fine, fibrous; typically white in color, actual color of the roots is dependent on substrate composition, water quality, fertilizers, substrate temperature and age of roots.
   Rooting habit.—Freely branching; dense.
Plant description:
   Plant and growth habit.—Compact, semi-upright and outwardly spreading to trailing and decumbent plant habit; freely branching habit with about nine primary lateral branches developing per plant; pinching enhances branching; dense and full appearance; vigorous growth habit; moderate growth rate.
   Plant height.—About 18.5 cm.
   Plant diameter.—About 63 cm.
Lateral branch description:
   Length.—About 30 cm.
   Diameter.—About 3 mm.
   Internode length.—About 2.7 cm.
   Strength.—Strong.
   Aspect.—Initially upright to outwardly spreading to trailing and decumbent.
   Texture.—Pubescent.
   Color.—Close to 144C and 195B.
Leaf description:
   Arrangement.—Before flowering, alternate, and after flowering, opposite; simple.
   Length.—About 3.2 cm.
   Width.—About 11.4 mm.
   Shape.—Oblanceolate.
   Apex.—Rounded.
   Base.—Cuneate.
   Margin.—Entire.
   Texture, upper and lower surfaces.—Pubescent.
   Luster, upper and lower surfaces.—Matte.
   Venation pattern.—Pinnate; arcuate.
   Color.—Developing leaves, upper surface: Close to 144A. Developing leaves, lower surface: Close to 144B. Fully expanded leaves, upper surface: Close to 137B, venation, close to 143C. Fully expanded leaves, lower surface: Close to 139C; venation, close to 143D.
   Petioles.—Length: About 5.2 mm. Diameter: About 1.7 mm. Texture, upper and lower surfaces: Pubescent. Color, upper surface: Close to 144C. Color, lower surface: Close to 144D.
Flower description:
   Flower arrangement and habit.—Single salverform flowers arising from leaf axils; freely flowering habit with usually about 320 flowers developing per plant; flowers face upright or outwardly.
   Fragrance.—None detected.
   Natural flowering season.—Early flowering habit, plants of the new Calibrachoa initiate and develop flowers about four to five weeks after planting; plants flower continuously from the spring throughout the summer in Germany.
   Flower longevity.—Individual flowers last about seven to ten days on the plant; flowers not persistent.
   Flower diameter.—About 3.4 cm.
   Flower length (height).—About 2.9 cm.
   Flower throat diameter.—About 9.4 mm.
   Flower tube length.—About 1.5 cm.
   Flower tube diameter, base.—About 2 mm.
   Flower buds.—Length: About 1.7 cm. Diameter: About 4.4 mm. Shape: Elongated oblong. Texture: Pubescent. Color: Close to 10B and 83A.
   Corolla.—Arrangement: Five petals fused at the base and opening into a flared trumpet. Petal length from throat: About 1.7 cm. Petal lobe width: About 1.7 cm. Petal shape: Roughly spatulate. Petal apex: Rounded. Petal margin: Entire. Petal texture, upper surface: Smooth, glabrous; velvety. Petal texture, lower surface: Smooth, glabrous. Throat texture: Smooth, glabrous. Tube texture: Smooth, glabrous. Color: Petal, when opening, upper surface: Close to 83A. Petal, when opening, lower surface: Close to 80B. Petal, fully opened, upper surface: Close to 81A; towards the base and venation, close to 79A; main color becoming closer to 81B with development. Petal, fully opened, lower surface: Close to 79D; venation, close to 78B. Throat: Close to 7A; venation, close to 79B. Tube: Close to 154C; venation, close to 187C.
   Calyx.—Arrangement: Star-shaped calyx with five sepals; sepals fused at the base. Sepal length: About 1.1 cm. Sepal width: About 2.8 mm. Sepal shape: Lanceolate. Sepal apex: Acute. Sepal margin: Entire. Sepal texture, upper and lower surfaces: Pubescent. Sepal luster, upper and lower surfaces: Matte. Color, upper surface: Close to 137B. Color, lower surface: Close to 137C.
   Peduncles.—Length: About 1.3 cm. Diameter: About 1.1 mm. Angle: About 45° from stem axis. Strength: Moderately strong. Texture: Pubescent. Color: Close to 144C.
   Reproductive organs.—Stamens: Quantity: Six per flower. Filament length: About 4.8 mm. Filament color: Close to 145D. Anther shape: Ellipsoidal. Anther length: About 1.5 mm. Anther color: Close to 8A. Pollen amount: Scarce. Pollen color: Close to 6B. Pistils: Quantity: One per flower. Pistil length: About 7.4 mm. Style length: About 5.4 mm. Style color: Close to 145D. Stigma shape: Ellipsoidal. Stigma color: Close to 144C. Ovary color: Close to 145B. Seeds and fruits: Seed and fruit development have not been observed on plants of the new Calibrachoa.
Garden performance: Plants of the new Calibrachoa have been observed to have good garden performance and tolerate wind, rain and temperatures ranging from about 5° C. to about 40° C.
Pathogen & pest resistance: Plants of the new Calibrachoa have not been observed to be resistant to pathogens and pests common to Calibrachoa plants.

It is claimed:

1. A new and distinct Calibrachoa plant named ‘Duecalindapu’ as illustrated and described.
